(19) United States
(12) Patent Application Publication
(10) Pub. No.: US 2026/0325599 A1
(43) Pub. Date: Oct. 22, 2026

(54) COMPOSITION FOR INHIBITING SKIN GLYCATION COMPRISING TEA TREE EXTRACT

(71) Applicant: AMOREPACIFIC CORPORATION, Seoul (KR)

(72) Inventors: Si Young CHO, Yongin-si (KR); Hee Young JEON, Yongin-si (KR); Kyeong Hwan HWANG, Yongin-si (KR); Yu Mi KANG, Yongin-si (KR); Shin Hyoung KIM, Yongin-si (KR); A Young KIM, Yongin-si (KR); Hyoung June KIM, Yongin-si (KR); Ji Yong JUNG, Yongin-si (KR); Yong Deog HONG, Yongin-si (KR)

(73) Assignee: AMOREPACIFIC CORPORATION, Seoul (KR)

(21) Appl. No.: 19/560,444

(22) Filed: Mar. 9, 2026

(30) Foreign Application Priority Data
Mar. 10, 2025 (KR) 10-2025-0030799

(51) Int. Cl. A61K 36/82; A61K 8/9789; A61K 31/353; A61Q 19/00; A61Q 19/08

(52) U.S. Cl. CPC A61K 36/82; A61K 8/9789; A61K 31/353; A61Q 19/007; A61Q 19/08

(57) ABSTRACT

The present specification relates to a composition comprising a Jangwon No. 3 tea plant extract as an active ingredient. The composition, by comprising the Jangwon No. 3 tea plant extract as the active ingredient, promotes an activity of a lysosome involved in removal of waste products in skin cells, and can provide an effect of inhibiting skin glycation and removing advanced glycation end products which are representative waste products. Through this, the yellowishness of the skin is removed, and thus the skin can be purified and a skin tone can be improved.

### CROSS REFERENCE TO RELATED APPLICATION

The present application claims priority to Korean Patent Application No. 10-2025-0030799, filed Mar. 10, 2025, the entire contents of which are hereby incorporated by this reference.

### BACKGROUND OF THE INVENTION

### Field of the Invention

The present specification describes a composition comprising a tea plant extract.

### Description of the Related Art

Inside skin cells, various waste products are accumulated by various external stimuli or aging. In these waste products, damaged proteins, glycation products, reactive oxygen species, and substances of viruses or bacteria are included. The intracellular waste products are accumulated in a lysosome performing a role as a recycling center, and to this, various degradative enzymes are combined, an autolysosome is created, and an action (autophagy) of removing the waste products occurs. If a function of the lysosome is deteriorated, the removal of the waste products is not performed, and thus the glycation products and the like are accumulated, toxicity is increased, and growth is inhibited. Due to this, because of the waste products accumulated in epidermal tissue, skin becomes dull, and yellowishness is seen to be increased. Also, by decreasing a turnover of the epidermal tissue, it is being reported that dead skin cells are increased, and a rough skin state without gloss is caused. To improve such a skin state, an exfoliant or the like is used, but the use of such an exfoliant has a very temporary effect, and upon long-term use, a risk of inducing damage of a skin barrier is very high. Particularly, for skin of the late 40s or older in which the skin turnover is slow, frequent use of the exfoliant may rather induce skin dryness and promote wrinkle formation. Therefore, by activating the waste product removal action of the skin cells themselves, a new skin purification action solution capable of improving the dullness and the yellowishness of the skin and improving skin texture without cell damage is required.

### SUMMARY OF THE INVENTION

In one aspect, the present disclosure has been made in an effort to provide a composition capable of improving dullness of skin by inhibiting skin glycation.

In one aspect, one embodiment of the present disclosure provides a composition for inhibiting skin glycation, comprising a Jangwon No. 3 tea plant extract as an active ingredient.

The composition according to one embodiment of the present disclosure, by comprising the Jangwon No. 3 tea plant extract among various tea plant varieties as the active ingredient, promotes an activity of a lysosome involved in removal of waste products in skin cells, and may provide an effect of inhibiting the skin glycation and removing advanced glycation end products (AGEs) which are representative waste products. Accordingly, an effect of improving yellowishness removal and improving stability may be provided. Through this, the yellowishness of the skin is removed, and thus the skin may be purified and a skin tone may be improved.

### BRIEF DESCRIPTION OF THE DRAWINGS

FIG. 1 is a view comparing a lysosome activity promotion effect of Example 1 (JW3) and Comparative Example 1 (GTE) according to one embodiment of the present disclosure through fluorescence images.

FIG. 2A is a view showing a graph comparing the lysosome activity promotion effect of Example 1 (JW3) and Comparative Example 1 (GTE) according to one embodiment of the present disclosure.

FIG. 2B is a view showing a graph comparing the lysosome activity promotion effect of Example 1 (JW3) according to one embodiment of the present disclosure by concentration.

FIG. 3 is a view comparing an autophagy activity promotion effect of Example 1 (JW3) and Comparative Example 1 (GTE) according to one embodiment of the present disclosure through fluorescence images.

FIG. 4 is a view showing a graph comparing the autophagy activity promotion effect of Example 1 (JW3) and Comparative Example 1 (GTE) according to one embodiment of the present disclosure.

FIG. 5A is a view showing a result of comparing an advanced glycation end products removal effect in aging-induced cells treated with Example 1 (JW3) and Comparative Example 1 (GTE) according to one embodiment of the present disclosure.

FIG. 5B is a view showing a result of comparing the advanced glycation end products removal effect in aging-induced cells treated with Example 1 (JW3) according to one embodiment of the present disclosure by treatment concentration.

### DETAILED DESCRIPTION OF THE INVENTION

Hereinafter, exemplary embodiments of the present disclosure will be described in detail with reference to the accompanying drawings.

The embodiments of the present disclosure disclosed herein are illustrated for purposes of description only, and the embodiments of the present disclosure may be practiced in various forms and should not be interpreted as limiting to the embodiments described herein. The present disclosure is subject to various modifications and may have various forms, and the embodiments are not intended to limit the present disclosure to any particular disclosure form, but are to be understood to include all modifications, equivalents, or substitutions that fall within the scope of the spirit and art of the present disclosure. Singular expressions include plural expressions unless clearly described as different meanings in the context. In the present application, the terms “comprises,” “comprising,” “includes,” “including,” “containing,” “has,” “having” or other variations thereof are inclusive and therefore specify the presence of stated features, integers, steps, operations, constituent elements, components (or parts), or combinations thereof, but do not exclude the presence or addition of one or more other features, integers, steps, operations, constituent elements, components (or parts), or combinations thereof.

One embodiment of the present disclosure may provide a composition for inhibiting skin glycation, comprising a Jangwon No. 3 tea plant extract as an active ingredient.

Another embodiment may provide a use of the Jangwon No. 3 tea plant extract for use in manufacture of a composition for inhibiting skin glycation. Another embodiment may provide a method for inhibiting skin glycation, comprising administering the Jangwon No. 3 tea plant extract in an effective amount to a subject in need thereof. Another embodiment may provide the Jangwon No. 3 tea plant extract as an active ingredient for use in a composition for inhibiting skin glycation. In addition, a non-therapeutic use of the Jangwon No. 3 tea plant extract as an active ingredient for inhibiting skin glycation may be provided.

In the present disclosure, the Jangwon No. 3 is a variety name of a new variety of Camellia sinensis, Theaceae, and a scientific name is Camellia sinensis L. The Jangwon No. 3 exhibits morphological characteristics that young shoots with one bud and three leaves and a length of a leaf blade are on a long side, a posture of the leaf blade is upward, an intensity of a color of the leaf blade is medium, and a position where a flower style splits is high. Compared with Yabukita, which is a variety generally used for a green tea extract, it exhibits morphological differences that a width of a leaf blade is narrow, a shape of the leaf blade is a narrower ellipse, and a serrated shape of a leaf blade edge is deep. In one embodiment, the Jangwon No. 3 may be KCTC (Korean Collection for Type Cultures) 12213BP deposited on May 14, 2012 with the Korea Research Institute of Bioscience and Biotechnology.

Also, the extract of the Jangwon No. 3 tea plant also exhibits differences in types and contents of constituent components from an extract of a general tea plant variety.

In one embodiment, the extract of the Jangwon No. 3 tea plant may be one obtained by processing buds or leaves of Jangwon No. 3 tea plant.

In one embodiment, the extract of the Jangwon No. 3 tea plant may comprise methylated catechin (epigallocatechin-3-O-(3-O-methyl)-gallate (EGCG3″Me)) in a high content of about 10 times or more than the extract of the general tea plant variety. Specifically, the Jangwon No. 3 tea plant extract may comprise the methylated catechin in 0.5 to 10 wt % relative to a total weight of the extract. More specifically, the Jangwon No. 3 tea plant extract may comprise the methylated catechin in 0.5 wt % or more, 1 wt % or more, 1.5 wt % or more, 2 wt % or more, 2.5 wt % or more, 3 wt % or more, 3.5 wt % or more, 4 wt % or more, 4.5 wt % or more, 5 wt % or more, 6 wt % or more, 7 wt % or more, 8 wt % or more, or 9 wt % or more relative to the total weight of the extract, and may be comprised in 10 wt % or less, 9 wt % or less, 8 wt % or less, 7 wt % or less, 6 wt % or less, 5 wt % or less, 4.5 wt % or less, 4 wt % or less, 3.5 wt % or less, 3 wt % or less, 2.5 wt % or less, 2 wt % or less, 1.5 wt % or less, or 1 wt % or less.

In one embodiment, the extract of the Jangwon No. 3 tea plant may further comprise one or more selected from the group consisting of gallocatechin, epigallocatechin, catechin, epicatechin, epigallocatechin gallate, gallocatechin gallate, and epicatechin gallate in addition to the methylated catechin.

In one embodiment, the extract of the Jangwon No. 3 tea plant may comprise 0.5 to 10 wt %, more specifically, 1 to 3 wt % of methylated catechin relative to the total weight of the extract. In one embodiment, the extract of the Jangwon No. 3 tea plant may comprise 0.41 to 1 wt %, more specifically, 0.45 to 0.7 wt % of gallocatechin relative to the total weight of the extract. In one embodiment, the extract of the Jangwon No. 3 tea plant may comprise 8 to 15 wt %, more specifically, 10 to 15 wt % of epigallocatechin relative to the total weight of the extract. In one embodiment, the extract of the Jangwon No. 3 tea plant may comprise 0.3 to 1 wt %, more specifically, 0.4 to 0.6 wt % of catechin relative to the total weight of the extract. In one embodiment, the extract of the Jangwon No. 3 tea plant may comprise 2.1 to 10 wt %, more specifically, 2.5 to 4.5 wt % of epicatechin relative to the total weight of the extract. In one embodiment, the extract of the Jangwon No. 3 tea plant may comprise 16 to 30 wt %, more specifically, 18 to 20 wt % of epigallocatechin gallate relative to the total weight of the extract. In one embodiment, the extract of the Jangwon No. 3 tea plant may comprise 0.11 to 0.5 wt %, more specifically, 0.15 to 0.4 wt % of gallocatechin gallate relative to the total weight of the extract. In one embodiment, the extract of the Jangwon No. 3 tea plant may comprise 3 to 15 wt %, more specifically, 4 to 10 wt % of epicatechin gallate relative to the total weight of the extract.

In one embodiment, the extract of the Jangwon No. 3 tea plant may be one obtained by processing first-flush tea leaves of Jangwon No. 3 tea plant.

Generally, for tea plant, when harvested once, leaves grow from one bud and one leaf to five leaves, and such a harvesting process is repeatedly performed about four times a year. When the leaves of tea plant are classified according to a harvesting period, these may be named first-flush tea, second-flush tea, third-flush tea, and fourth-flush tea. Specifically, the first-flush tea may be one in which tea leaves are harvested for a first time by May of a corresponding year, the second-flush tea may be one harvested for a second time after harvesting the first-flush tea of the corresponding year (harvested in June to July), the third-flush tea may be one harvested for a third time after harvesting the second-flush tea of the corresponding year (harvested in August), and the fourth-flush tea may be one harvested for a fourth time after harvesting the third-flush tea of the corresponding year (harvested on or after September). Also, when the leaves of tea plant are classified according to leaf growth, they may be divided into a first leaf to a fifth leaf according to an order in which leaves emerge from one tea plant (one bud), and it takes about 30 days to 35 days until becoming the fifth leaf after sprouting.

From the above aspect, the ‘first-flush tea’ according to one embodiment may mean one in which tea leaves are harvested for the first time by May of the corresponding year. The ‘first-flush tea’ according to one embodiment may mean one in which tea leaves are harvested at a one-bud and five-leaf stage. For example, the extract of the Jangwon No. 3 tea plant may be one in which the upper first leaf to third leaf of a new shoot are collected based on a period when an opening degree of the one-bud and five-leaf stage is 70% during a first-flush tea period of the Jangwon No. 3 tea plant. In the present disclosure, general green tea corresponding to the first-flush tea means one using tea leaves collected at remaining periods excluding the first-flush tea.

In one embodiment, the Jangwon No. 3 tea plant extract may be an aqueous ethanol solution extract of Jangwon No. 3 tea plant leaves.

In one embodiment, the Jangwon No. 3 tea plant extract may be one manufactured by an extraction method comprising: a step of steaming the tea leaves by steaming and then drying; a step of pulverizing and powdering a dried product obtained in the step; a step of extracting the powdered tea leaves using an aqueous ethanol solution as an extraction solvent; and a step of filtering an extract obtained in the extraction step and then concentrating under reduced pressure to obtain an extract. In one embodiment, the steaming by steaming may be steaming by passing the tea leaves through a steamer for 20 to 60 seconds. In one embodiment, the concentration under reduced pressure may be performed at 40 to 45° C.

In one embodiment, the composition may comprise the Jangwon No. 3 tea plant extract in 0.0001 to 50 wt % relative to a total weight of the composition. Specifically, the composition may comprise the Jangwon No. 3 tea plant extract in 0.0001 wt % or more, 0.001 wt % or more, 0.01 wt % or more, 0.1 wt % or more, 1 wt % or more, 5 wt % or more, 10 wt % or more, 15 wt % or more, 20 wt % or more, 25 wt % or more, 30 wt % or more, 35 wt % or more, 40 wt % or more, or 45 wt % or more relative to a total weight of the composition, and may comprise the same in 50 wt % or less, 45 wt % or less, 40 wt % or less, 35 wt % or less, 30 wt % or less, 25 wt % or less, 20 wt % or less, 15 wt % or less, 10 wt % or less, 5 wt % or less, 1 wt % or less, or 0.1 wt % or less.

In one embodiment, the Jangwon No. 3 tea plant extract promotes an activity of a lysosome in skin cells, and by activating a waste product removal action such as advanced glycation end products of the skin cells themselves, dullness and yellowishness of skin may be improved and skin texture may be improved without cell damage.

From the above aspect, the composition according to one embodiment may be for removal of waste products in skin. Another embodiment may provide a use of the Jangwon No. 3 tea plant extract for use in manufacture of a composition for removal of waste products in skin. Another embodiment may provide a method for removal of waste products in skin, comprising administering the Jangwon No. 3 tea plant extract in an effective amount to a subject in need thereof. Another embodiment may provide the Jangwon No. 3 tea plant extract as an active ingredient for use in a composition for removal of waste products in skin. In addition, a non-therapeutic use of the Jangwon No. 3 tea plant extract as an active ingredient for removal of waste products in skin may be provided.

Also, the composition according to one embodiment may be for skin tone improvement. Another embodiment may provide a use of the Jangwon No. 3 tea plant extract for use in manufacture of a composition for skin tone improvement. Another embodiment may provide a method for skin tone improvement, comprising administering the Jangwon No. 3 tea plant extract in an effective amount to a subject in need thereof. Another embodiment may provide the Jangwon No. 3 tea plant extract as an active ingredient for use in a composition for skin tone improvement. In addition, a non-therapeutic use of the Jangwon No. 3 tea plant extract as an active ingredient for skin tone improvement may be provided.

The composition according to one embodiment may be for skin purification. Another embodiment may provide a use of the Jangwon No. 3 tea plant extract for use in manufacture of a composition for skin purification. Another embodiment may provide a method for skin purification, comprising administering the Jangwon No. 3 tea plant extract in an effective amount to a subject in need thereof. Another embodiment may provide the Jangwon No. 3 tea plant extract as an active ingredient for use in a composition for skin purification. In addition, a non-therapeutic use of the Jangwon No. 3 tea plant extract as an active ingredient for skin purification may be provided.

In one embodiment, the composition may be targeted for the 40s or 45 years of age or older in which skin turnover is slow.

In one embodiment, the composition according to one embodiment of the present disclosure may be a cosmetic composition. The cosmetic composition may be formulated by containing a cosmetologically or dermatologically acceptable medium or base. This refers to all formulations suitable for topical application, and may be for transdermal administration. For example, it may be provided in a form of a solution, a gel, a solid, a paste anhydrous product, an emulsion obtained by dispersing an oil phase in an aqueous phase, a suspension, a microemulsion, microcapsules, microgranules, or ionic (liposome) and non-ionic vesicle dispersants, or in a form of a cream, a toner, a lotion, a powder, an ointment, a spray, or a concealer stick. Also, it may be used in a form of a foam or in a form of an aerosol composition further containing a compressed propellant. As another example, it may be a formulation of an injectable such as a filler, a patch, and the like, but is not limited thereto. These compositions may be manufactured according to conventional methods in the art.

As one embodiment, the composition according to one embodiment of the present disclosure, in addition to the active ingredient, may comprise other components capable of preferably giving a synergistic effect to a main effect within a range that does not damage the main effect, and in addition to the active ingredient of the present disclosure, other components may be appropriately selected and blended by those skilled in the art without difficulty according to a formulation or a purpose of use of other cosmetic compositions. Also, as one embodiment, the cosmetic composition of the present disclosure, in addition to the above components, may comprise other components usually blended in a cosmetic composition as necessary. For example, a moisturizer, an emollient, organic and inorganic pigments, organic powder, a UV absorber, a preservative, a bactericide, an antioxidant, a plant extract, a pH adjuster, alcohol, colorants, fragrance, a blood circulation promoter, a cooling agent, an antiperspirant, purified water, and the like may be mentioned. Other blending components capable of being comprised in the cosmetic composition of the present disclosure are not limited thereto, and a blending amount of the components is possible within a range that does not damage the purpose and effect of the present disclosure.

The composition according to one embodiment of the present disclosure may be an oral composition comprising the active ingredient. As one embodiment, the composition may be a food composition. For example, it may be processed into functional foods such as fermented milk, cheese, yogurt, juice, probiotic preparations, and health foods comprising the active ingredient, and may be used in a form of various other food additives. As one embodiment, the composition may be a composition for health food. As one embodiment, the composition for health food may be formulated into a pill, a capsule, a tablet, granules, a caramel, or a drink, or the like. As another embodiment, it may also be processed in a form of a liquid, powder, granules, a tablet, or a tea bag, or the like. The composition may be administered by various methods such as simple drinking, injection administration, a spray method, or a squeeze method. The composition may comprises other components and the like capable of giving a synergistic effect to a main effect within a range that does not damage the main effect of the present disclosure. For example, to improve physical properties, additives such as fragrance, a colorant, a bactericide, an antioxidant, a preservative, a moisturizer, a thickener, inorganic salts, an emulsifier, and a synthetic polymer substance may be further comprised. In addition to that, auxiliary components such as water-soluble vitamins, oil-soluble vitamins, high molecular peptides, high molecular polysaccharides, and seaweed extract may be further comprised. The components may be appropriately selected and blended by those skilled in the art according to a formulation or a purpose of use, and an addition amount thereof may be selected within a range that does not damage the purpose and effect of the present disclosure. For example, the addition amount of the components may be 0.0001 wt % to 99.9 wt % based on a total weight of the composition.

Hereinafter, the present disclosure will be described in detail with reference to Examples, Comparative Examples, and Test Examples. It will be obvious to those having ordinary knowledge in the art that these are merely presented illustratively to describe the present disclosure more specifically, and a scope of the present disclosure is not limited by these Examples, Comparative Examples, and Test Examples.

### [Example 1] Manufacture of Jangwon No. 3 Tea Plant Extract

Jangwon No. 3 (Osulloc Farm Co., Ltd., Agricultural Corporation) was collected from the upper first leaf to third leaf of a new shoot based on a period when an opening degree of a one-bud and five-leaf stage was 70% during a first-flush tea (early May) period, and was steamed by passing through a steamer for 40 seconds, dried in a dryer at 80° C., and then powder-processed by a pulverizer, sieved through a 60-mesh sieve, and powdered. To 100 g of the pulverized sample of the Jangwon No. 3 thus manufactured, 1 L of a 70% (v/v) aqueous ethanol solution (anhydrous ethanol 70% (v/v)+distilled water 30% (v/v)) was added, and after performing reflux extraction and filtering, it was concentrated under reduced pressure at 40 to 45° C., and finally 19.2 g of a dried extract of leaves of a new variety of tea plant (Jangwon No. 3) was secured.

### Comparative Example 1

A green tea extract from a general tea plant was manufactured in the same method as in Example 1, except that a variety of tea plant was Yabukita (Samkyung Costech).

### [Test Example 1] Comparison of Catechin Composition Comprised in Tea Plant Extract

For a comparative analysis of catechin components comprised in the tea plant extracts of Example 1 and Comparative Example 1 manufactured above, the following experiment was performed.

After dissolving each of the extracts in 50% methanol to make a 10,000 ppm solution, component analysis (Waters Co., 2695/2996 PDA detector) was performed using high-performance liquid chromatography (HPLC) (Waters Co.). For a stationary phase, a Thermo Syncronis C18 (4.6×250 mm, 5 μm), P/N: 97105-254630 column was used, and for a mobile phase, a composition ratio as shown in Table 1 below was used.

TABLE 1

| Time (minutes) | A: 0.1% Acetic acid in water | B: Acetonitrile |
|---|---|---|
| 0 | 90 | 10 |
| 10.0 | 90 | 10 |
| 30.0 | 85 | 15 |
| 53.0 | 80 | 20 |
| 53.1 | 5 | 95 |
| 55.0 | 5 | 95 |
| 55.1 | 90 | 10 |
| 60.0 | 90 | 10 |

As shown in Table 2 below, it was confirmed that the extract of Jangwon No. 3 leaves comprises epigallocatechin-3-O-(3-O-methyl) gallate (EGCG3″Me; 3″-O-Me-EGCG) in an amount of 2.5 wt % based on a total weight of the extract, which is comprised in a high content of 10 times or more than a general tea plant variety.

TABLE 2

| | | Comparative |
|---|---|---|
| | | Example 1 |
| | Example 1 | (General |
| | (Jangwon No. 3 | green tea |
| Item | extract) | extract) |
| (1) gallocatechin (GC) | 0.5 | 0.4 |
| (2) epigallocatechin (EGC) | 12.7 | 7.7 |
| (3) catechin (C) | 0.5 | 0.2 |
| (4) epicatechin (EC) | 3.1 | 2.0 |
| (5) epigallocatechin gallate (EGCG) | 18.8 | 15.1 |
| (6) gallocatechin gallate (GCG) | 0.2 | 0.1 |
| (7) epicatechin gallate (ECG) | 5.3 | 2.6 |
| (8) catechin gallate (CG) | N/D (Not | N/D |
| | Detected) | |
| Total content of 8 types of catechins | 41.1 | 28.1 |
| 3″-O-Me-EGCG content | 2.5 | 0.2 |

(wt %)

### [Test Example 2] Lysosome and Autophagy Activity Analysis

To confirm a lysosome and autophagy activity improvement effect of the extracts of Example 1 and Comparative Example 1, the following experiment was performed.

First, aging-induced skin keratinocytes were prepared. Specifically, skin keratinocytes (normal human epidermal keratinocytes, NHEK) were seeded to be 12,000 cells/cm² and cultured until reaching 80% confluency. To the cell culture, UVB 20 mJ/cm² was repeatedly irradiated once a day for 3 days, and cultured for 5 days. After 5 days, it was confirmed that aging was induced by measuring activities of Lamin B1, which is an aging-related marker, and senescence-associated beta-galactosidase.

### Measurement of Lysosome Activity

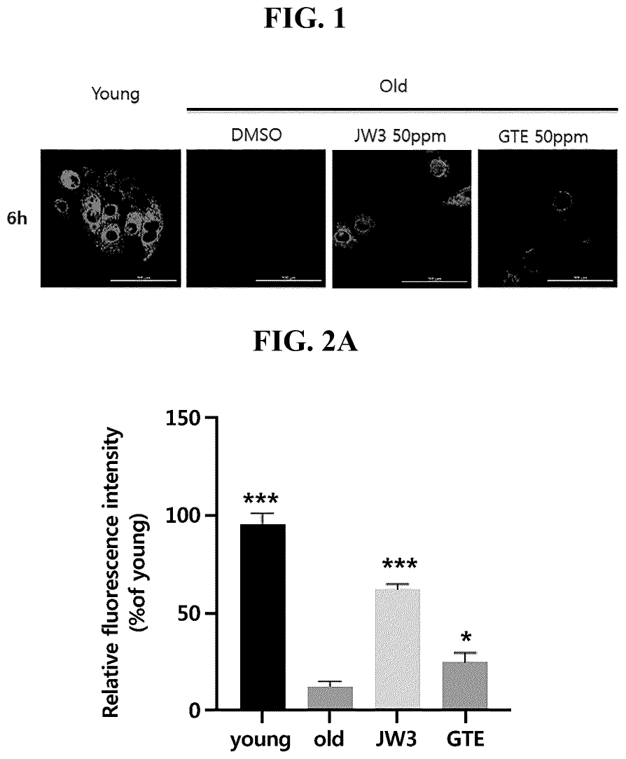

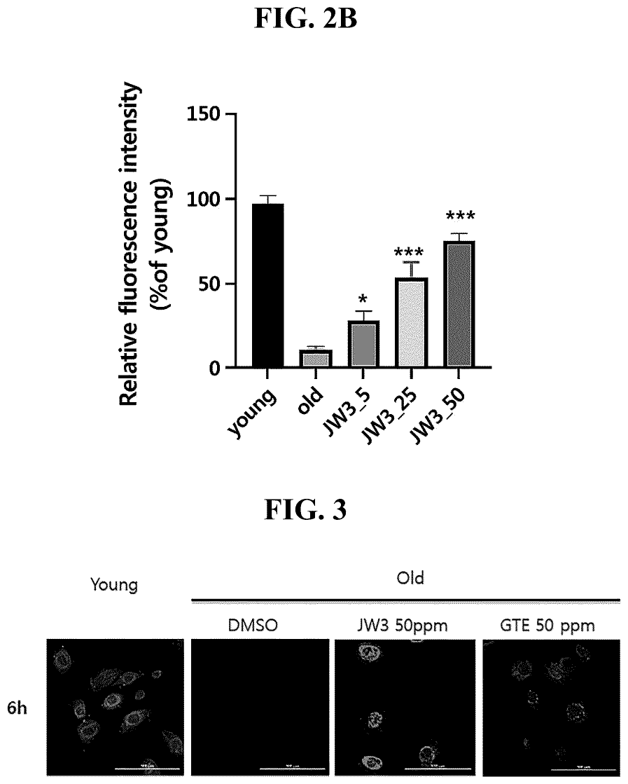

The aging-induced skin keratinocytes were treated with 50 ppm of Jangwon No. 3 extract (Example 1), 50 ppm of general green tea extract (Comparative Example 1), or DMSO (dimethyl sulfoxide, control group) for 24 hours. Next, the cells were treated with 75 nM of LysoTracker Deep Red (molecular probe, life technologies) and reacted for 6 hours, and then this was confirmed under a confocal microscope and shown in FIG. 1, and a result of red fluorescence intensity analysis is shown in FIG. 2A.

Also, the same experiment was performed by varying a treatment concentration of the Jangwon No. 3 extract (Example 1), and a result thereof is shown in FIG. 2B.

### Measurement of Autophagy Activity

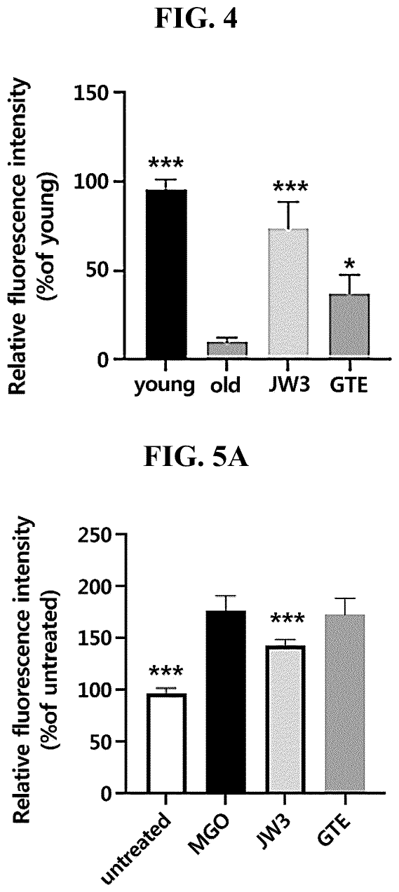

The aging-induced skin keratinocytes were treated with 50 ppm of Jangwon No. 3 extract (Example 1), 50 ppm of general green tea extract (Comparative Example 1), or DMSO (dimethyl sulfoxide, control group) for 24 hours. The cells were treated with 1 mM DALGreen (Dojindo) and reacted for 30 minutes, and then, after exchanging with a new medium, a drug was treated for 6 hours and this was confirmed under a confocal microscope and shown in FIG. 3, and a result of green fluorescence intensity analysis is shown in FIG. 4.

As a result, as shown in FIG. 1 to FIG. 4, compared with the control group (DMSO) in which aging is induced and lysosome and autophagy activities are decreased, a large change did not appear upon treatment of the general green tea extract (Green tea extract (GTE), Comparative Example 1), whereas it can be confirmed that for the Jangwon No. 3 extract (JW3, Example 1) according to one embodiment of the present disclosure, both lysosome and autophagy activities were recovered to a level close to skin keratinocytes (Young) in which aging was not induced. This means that the Jangwon No. 3 tea plant extract, which is the active ingredient of the present disclosure, has an effect of improving the autophagy activity decreased by aging which does not appear in the general green tea extract, and accordingly, an anti-glycation, that is, a skin glycation inhibition effect, may be provided.

### [Test Example 3] Verification of Advanced Glycation End Products Removal Effect

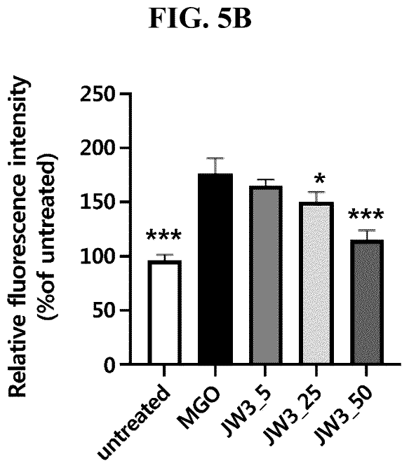

To the aging-induced skin keratinocytes prepared in Test Example 2, the Jangwon No. 3 extract (Example 1) or the general green tea extract (Comparative Example 1) was treated with 500 mM methylglyoxal (MGO) (Sigma), which is a glycation inducing substance, for 3 days, and then fluorescence was measured at Ex 370 nm/Em 440 nm and Ex 335/Em 385 nm using a microplate reader to confirm whether advanced glycation end products were accumulated, and this is shown in FIG. 5A. Also, the same experiment was performed by varying the treatment concentration of the Jangwon No. 3 extract (Example 1), and a result thereof is shown in FIG. 5B.

As a result, as shown in FIG. 5A and FIG. 5B, compared with the control group (MGO) in which aging is induced and anti-glycation activity is decreased, the advanced glycation end products were hardly removed upon treatment of the general green tea extract (Green tea extract (GTE), Comparative Example 1), whereas it can be confirmed that upon treatment of the Jangwon No. 3 extract (JW3, Example 1) according to one embodiment of the present disclosure, the advanced glycation end products were removed and a fluorescence intensity was significantly decreased. This means that the Jangwon No. 3 tea plant extract, which is the active ingredient of the present disclosure, has a skin glycation inhibition effect which does not appear in the general green tea extract, and accordingly, a skin purification and skin tone improvement effect may be provided by removing the advanced glycation end products, which are skin waste products remaining in aging skin.

Formulation Examples of the composition according to one embodiment of the present specification are described below, but application to various other formulations is also possible, and this is not intended to limit the present specification but merely to describe it specifically.

### [Formulation Example 1] Softening Toner (Skin Lotion)

A softening toner was manufactured by a conventional method according to a composition described in the table below.

TABLE 3

| Blending component | Content (wt %) |
|---|---|
| Jangwon No. 3 tea plant extract | 10 |
| Glycerin | 3.0 |
| Butylene glycol | 2.0 |
| Propylene glycol | 2.0 |
| Carboxyvinyl polymer | 0.1 |
| PEG-12 nonylphenyl ether | 0.2 |
| Polysorbate 80 | 0.4 |
| Ethanol | 10.0 |
| Triethanolamine | 0.1 |
| Preservative, colorant, fragrance | q.s. |
| Purified water | balance |

### [Formulation Example 2] Nourishing Toner (Milk Lotion)

A nourishing toner was manufactured by a conventional method according to a composition described in the table below.

TABLE 4

| Blending component | Content (wt %) |
|---|---|
| Jangwon No. 3 tea plant extract | 10 |
| Glycerin | 3.0 |
| Butylene glycol | 3.0 |
| Propylene glycol | 3.0 |
| Carboxyvinyl polymer | 0.1 |
| Beeswax | 4.0 |
| Polysorbate 60 | 1.5 |
| Caprylic/Capric triglyceride | 5.0 |
| Squalane | 5.0 |
| Sorbitan sesquioleate | 1.5 |
| Liquid paraffin | 0.5 |
| Cetearyl alcohol | 1.0 |
| Triethanolamine | 0.2 |
| Preservative, colorant, fragrance | q.s. |
| Purified water | balance |

### [Formulation Example 3] Massage Cream

A massage cream was manufactured by a conventional method according to a composition described in the table below.

TABLE 5

| Blending component | Content (wt %) |
|---|---|
| Jangwon No. 3 tea plant extract | 10 |
| Glycerin | 8.0 |
| Butylene glycol | 4.0 |
| Liquid paraffin | 45.0 |
| Beta-glucan | 7.0 |
| Carbomer | 0.1 |
| Caprylic/Capric triglyceride | 3.0 |
| Beeswax | 4.0 |
| Cetearyl glucoside | 1.5 |
| Sorbitan sesquioleate | 0.9 |
| Vaseline | 3.0 |
| Paraffin | 1.5 |
| Preservative, colorant, fragrance | q.s. |
| Purified water | balance |

### [Formulation Example 4] Drink

After mixing 50 mg of Jangwon No. 3 tea plant extract, 10 g of glucose, 0.6 g of citric acid, and 25 g of liquid oligosaccharide, 300 ml of purified water is added and each bottle is filled with 200 ml. After filling the bottles, sterilization was performed at 130° C. for 4 to 5 seconds to manufacture a drink.

### [Formulation Example 5] Caramel Formulation

50 mg of Jangwon No. 3 tea plant extract, 1.8 g of corn syrup, 0.5 g of skim milk, 0.5 g of soy lecithin, 0.6 g of butter, 0.4 g of vegetable hydrogenated oil, 1.4 g of sugar, 0.58 g of margarine, and 20 mg of salt were mixed and caramel-molded.

### [Formulation Example 6] Health Food

TABLE 6

| Component | Content |
|---|---|
| Jangwon No. 3 tea plant extract | 100 |

Vitamin mixture

| | | |
|---|---|---|
| Vitamin A acetate | 70 | μg |
| Vitamin E | 1.0 | mg |
| Vitamin B1 | 0.13 | mg |
| Vitamin B2 | 0.15 | mg |
| Vitamin B6 | 0.5 | mg |
| Vitamin B12 | 0.2 | μg |
| Vitamin C | 10 | mg |
| Biotin | 10 | μg |
| Nicotinamide | 1.7 | mg |
| Folic acid | 50 | μg |
| Calcium pantothenate | 0.5 | mg |

Mineral mixture

| | | |
|---|---|---|
| Ferrous sulfate | 1.75 | mg |
| Zinc oxide | 0.82 | mg |
| Magnesium carbonate | 25.3 | mg |
| Monopotassium phosphate | 15 | mg |
| Dicalcium phosphate | 55 | mg |
| Potassium citrate | 90 | mg |
| Calcium carbonate | 100 | mg |
| Magnesium chloride | 24.8 | mg |

A composition ratio of the vitamin and mineral mixture was mixed and composed by taking components relatively suitable for health food as an example, but a blending ratio thereof may be arbitrarily modified and implemented, and after mixing the above components according to a conventional health food manufacturing method, granules are manufactured, and it may be used for manufacture of a health food composition according to a conventional method.

### [Formulation Example 7] Health Drink

TABLE 7

| Component | Content | |
|---|---|---|
| Jangwon No. 3 tea plant extract | 10 | mg |
| Citric acid | 1000 | mg |
| Oligosaccharide | 100 | g |
| Plum concentrate | 2 | g |
| Taurine | 1 | g |
| Purified water | balance | |
| Total volume | 900 | m [symbol] |

The remaining amount of purified water was added so that a total volume becomes 900 ml as in the table above, and after mixing the above components according to a conventional health drink manufacturing method, and then stirring and heating at 85° C. for about 1 hour, a produced solution is filtered and obtained in a sterilized 2 L container, sealed and sterilized, and then stored under refrigeration to be used for manufacture of a health drink composition.

### DEPOSIT NUMBER

Name of Depositary Institution: Korean Collection for Type Cultures (KCTC), Korea Research Institute of Bioscience and Biotechnology
Deposit number: KCTC12213BP
Deposit date: May 14, 2012

What is claimed is:

1. A method for inhibiting skin glycation, comprising administering a Jangwon No. 3 tea plant extract in an effective amount to a subject in need thereof.

2. The method of claim 1, wherein the extract comprises methylated catechin (epigallocatechin-3-O-(3-O-methyl)-gallate) in 0.5 to 10 wt % relative to a total weight of the extract.

3. The method of claim 2, wherein the extract further comprises one or more selected from the group consisting of gallocatechin, epigallocatechin, catechin, epicatechin, epigallocatechin gallate, gallocatechin gallate, and epicatechin gallate.

4. The method of claim 3, wherein the extract comprises, relative to the total weight of the extract,
0.5 to 10 wt % of methylated catechin;
0.41 to 1 wt % of gallocatechin;
8 to 15 wt % of epigallocatechin;
0.3 to 1 wt % of catechin;
2.1 to 10 wt % of epicatechin;
16 to 30 wt % of epigallocatechin gallate;
0.11 to 0.5 wt % of gallocatechin gallate; and
3 to 15 wt % of epicatechin gallate.

5. The method of claim 1, wherein the Jangwon No. 3 tea plant extract is an aqueous ethanol solution extract of Jangwon No. 3 tea plant leaves.

6. The method of claim 1, wherein the Jangwon No. 3 tea plant extract is comprised in a composition as an active ingredient, and the active ingredient is comprised in 0.0001 to 50 wt % relative to a total weight of the composition.

7. The method of claim 1, wherein the method is for removal of waste products in skin through inhibiting skin glycation.

8. The method of claim 1, wherein the method is for skin tone improvement through inhibiting skin glycation.

9. The method of claim 1, wherein the method is for skin purification through inhibiting skin glycation.

10. The method of claim 1, wherein the Jangwon No. 3 tea plant extract is comprised in a composition as an active ingredient, and the composition is a cosmetic composition.

11. The method of claim 1, wherein the Jangwon No. 3 tea plant extract is comprised in a composition as an active ingredient, and the composition is a food composition.
